(19) United States
(12) Patent Application Publication
(10) Pub. No.: US 2014/0371866 A1
(43) Pub. Date: Dec. 18, 2014

(54) TIBIAL IMPLANT DEVICES, SYSTEMS, AND METHODS

(71) Applicant: ConforMIS, Inc., Bedford, MA (US)

(72) Inventors: Nam T. Chao, Marlborough, MA (US); John Slamin, Wrentham, MA (US); Philipp Lang, Lexington, MA (US)

(21) Appl. No.: 14/373,555

(22) PCT Filed: Feb. 7, 2013
(86) PCT No.: PCT/US2013/025117

§ 371 Date: Jul. 21, 2014

Related U.S. Application Data
(60) Provisional application No. 61/596,209, filed on Feb. 7, 2012.

(51) Int. Cl. A61F 2/38

(52) U.S. Cl. CPC A61F 2/389
USPC 623/20.32

(57) ABSTRACT

Various embodiments described herein can facilitate the design of patient-adapted (e.g., patient-specific or patient-engineered) tibial implants. Furthermore, various embodiments described herein can include implant components having one or more patient-adapted features and one or more standard, that is, not patient-adapted (e.g., off-the-shelf), features incorporated into the design of the tibial implant components, including the sizes of the tibial tray, the locking mechanism, the tibial tray cavities, and/or the tibial inserts. For example, in various embodiments, the size of the tibial tray may be designed as patient-adapted by incorporating patient-adapted measurements into the perimeter of the tibial component.

### RELATED APPLICATIONS

This application claims the benefit of U.S. Provisional Patent Application No. 61/596,209 to Chao et al., entitled “Advanced Methods, Techniques, Devices and Systems for Securing Inserts into Tibial Tray Implants,” filed Feb. 7, 2012, the entire contents of which is incorporated herein by reference in its entirety.

### TECHNICAL FIELD

The present disclosure relates to articular repair systems (e.g., resection cut strategy, guide tools, and implant components) as described in, for example, U.S. patent application Ser. No. 13/397,457, entitled “Patient-Adapted and Improved Orthopedic Implants, Designs And Related Tools,” filed Feb. 15, 2012, and published as U.S. Patent Publication No. 2012-0209394, which is incorporated herein by reference in its entirety. In particular, the present disclosure relates to tibial implants.

### BACKGROUND

When a patient's knee is severely damaged, such as by osteoarthritis, rheumatoid arthritis, or post-traumatic arthritis, it may be desirous to repair and/or replace portions or the entirety of the knee with total or partial knee replacement implants. Knee replacement surgery, also known as knee arthroplasty, can help relieve pain and restore function in injured and/or severely diseased knee joints, and is a well-tolerated and highly successful procedure. Where a total or partial joint replacement is performed, it can be performed by a surgeon via an open procedure, with the distal end of the femur and the proximal end of the tibia exposed, and portions of the ends of these bones prepared by resecting bone surfaces of the tibia and femur.

Once the bone preparation has been completed, both the tibia and femur may receive artificial joint components, often made of metal alloys, high-grade plastics and/or polymers, to replace native anatomy. In the case of tibial artificial joint components, a tibial implant can include a receiver tray, typically made of metal, which is firmly fixed to the proximal tibia. In many cases, the tibial implant further includes an insert (also referred to as a “spacer” or “poly”), which is often made of flexible polyethylene or other polymer. The insert typically engages the tray and is positioned between the femoral component(s) and the tibial tray. The characteristics of engagement between the tray and insert can affect the quality of the insert fixation as well as the ease of insertion of the insert into the tray.

Accordingly, there is a need for improved tibial implants.

### BRIEF DESCRIPTION OF THE DRAWINGS

FIG. 1 illustrates exemplary embodiments of tibial implant designs;

FIG. 2 illustrates the side view of a total knee joint replacement assembly that includes one embodiment of a total knee femoral implant, tibial insert, and tibial tray;

FIG. 3 illustrates the front view of a total knee joint replacement assembly that includes the total knee femoral implant, tibial insert, and tibial tray;

FIG. 4 illustrates the back view of a total knee joint replacement assembly that includes the total knee femoral implant, tibial insert, and tibial tray;

FIG. 5 illustrates a separated front view of a total knee joint replacement assembly that includes the total knee femoral implant, tibial insert, and tibial tray;

FIG. 6 illustrates a separated, isometric front view of a total knee joint replacement assembly that includes the total knee femoral implant, tibial insert, and tibial tray;

FIG. 7 illustrates a side view of one exemplary tibial insert and tibial tray assembly;

FIG. 8 illustrates the back view of a tibial insert and tibial tray assembly;

FIG. 9 illustrates the front view of a tibial tray insert and tibial tray assembly;

FIG. 10 illustrates the front view of a tibial tray;

FIG. 11 illustrates the isometric view of a tibial tray;

FIG. 12 illustrates the side view of a tibial tray;

FIG. 13 illustrates the top view of a tibial tray;

FIG. 14 illustrates the back view of a tibial insert;

FIG. 15 illustrates the front view of a tibial insert;

FIG. 16 illustrates the side view of a tibial insert;

FIG. 17 illustrates an isometric bottom view of a tibial insert;

FIG. 18 illustrates the bottom view of a tibial insert;

FIG. 19 illustrates an isometric top view of a tibial tray;

FIG. 20 illustrates the top view of a tibial insert;

FIG. 21 illustrates a bottom plan view of insertion of inserts onto a tibial tray, with portions of the tibial tray depicted in shadow; and

FIG. 22 illustrates one exemplary embodiment of a tibial tray blank.

### DETAILED DESCRIPTION

“Tibial implant,” as used herein, can refer to an implant made of a single component or to an implant made of multiple components (e.g., tibial tray, insert(s)).

Various embodiments described herein can facilitate the design of patient-adapted (e.g., patient-specific or patient-engineered) tibial implants. Furthermore, various embodiments described herein can include implant components having one or more patient-adapted features and one or more standard, that is, not patient-adapted (e.g., off-the-shelf), features incorporated into the design of the tibial implant components, including the sizes of the tibial tray, the locking mechanism, the tibial tray cavities, and/or the tibial inserts. For example, in various embodiments, the size of the tibial tray may be designed as patient-adapted by incorporating patient-adapted measurements into the perimeter of the tibial component. A patient-adapted perimeter of the tibial component can be achieved, for example, by cutting the perimeter of a selected tibial component (such as a pre-manufactured blank component) to match the patient's cortical bone perimeter (or other anatomical features) in one or more dimensions of one more sections. Patient image data (as well as data derived from patient-specific data, including patient-engineered data) can be used to specifically design the perimeter of the tibial tray to create a unique patient-adapted size for the patient. In addition, a database of patient image data may be evaluated and statistically analyzed to create several standard “blank” sizes to be available for use with the most common patient size ranges. The standard blank sizes may be kept in inventory until needed, and then modified (if necessary) and shipped for a scheduled surgery.

Additionally or alternatively, some tibial implant embodiments may be designed specifically to include posterior-stabilization and/or accommodate posterior cruciate sacrifice and/or preservation. For posterior-stabilized designs, the posterior cruciate ligament and/or other soft tissue structures may be removed during the surgical procedure for a variety of reasons, including where such removal is necessitated due to injury and/or to accommodate a femoral or tibial implant component. In such embodiments, the tibial implant may be designed to have a post that can fit into a box and/or engage a bar or cam in a corresponding femoral implant component. This design can potentially replace and/or simulate the posterior cruciate ligament function, including preventing the femur from sliding forward too far on the tibia during knee flexion and/or extension. In other embodiments, the tibial implant may be designed for retaining or preserving cruciate ligament(s), which may include retention of the posterior cruciate ligament. This design may include a “deep dish” dimension on the posterior lip of the tibial implant (tray and/or the insert(s)). A higher wall or lip may be used to prevent sliding forward of a bone during knee motion

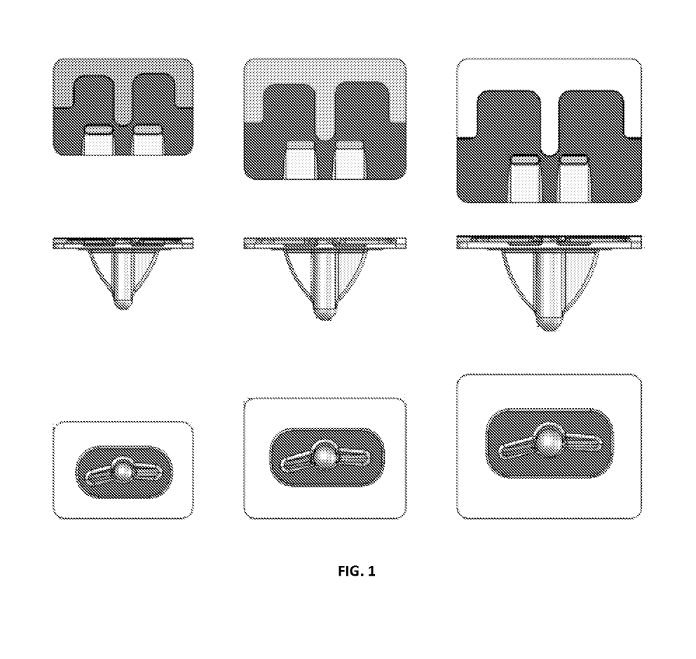

FIG. 1 depicts exemplary embodiments of a tibial implant design that incorporate one or more locking mechanisms to secure a tibial insert into a tibial tray. In these mechanisms, a corresponding lower surface on the tibial insert (not shown) can engage one or more ridges and/or recesses on the surface of the tibial tray, thereby locking the tibial insert in a desired position relative to the tray. The locking mechanism can be pre-configured and/or available, for example, in two or three different geometries or size. Optionally, a user or a computer program can have a library of CAD files or subroutines with different sizes and geometries of locking mechanisms available. For example, in a first step, the user or computer program can define, design or select a tibial, acetabular or glenoid implant profile that best matches a patient's cut (or, optionally, uncut) tibia, acetabulum or glenoid. In a second step, the user or computer program can then select the pre-configured CAD file or subroutine that is best suited for a given tibial or acetabular or glenoid perimeter or other shape or location or size. Moreover, various types of locking mechanisms (e.g., snap, dovetail, detent, protrusion captured by a recess) can be used. In some embodiments, the type of locking mechanism can be selected based on patient specific parameters, e.g. body weight, height, gender, race, activity level etc.).

In various embodiments, one or more locking mechanisms may be adapted to the patient's specific anatomy in at least one or more dimensions, as well as in all dimensions. In some embodiments, the location of locking features can be patient-adapted while the locking feature dimensions can be fixed. Alternatively, the locking mechanism can be pre-fabricated; in this embodiment, the location and dimensions of the locking mechanism may also be considered in the selection of the pre-fabricated components, so that any adaptations to the metal or ceramic backing relative to the patient's articular anatomy desirably do not compromise the locking mechanism. Accordingly, the components can be selected so that after adaptation to the patient's unique anatomy a minimum material thickness of the metal or ceramic backing comprising the tibial tray will be maintained adjacent to the locking mechanism and/or one or more desired perimeter features of a sufficient thickness are retained to allow the locking mechanism to function in a desired manner.

In some embodiments, the locking mechanism(s) for securing the tibial insert to the tibial tray can be designed and manufactured as an integral portion of the tibial tray. In some embodiments, the locking mechanism can be significantly smaller than the superior (also referred to herein as “upper”) surface of the tray, to allow for perimeter matching of the tray, whereby subsequent machining and/or processing of the outer periphery and superior portion of the tibial tray (e.g., to patient-matched or other desired dimensions) will not significantly degrade or otherwise affect the locking mechanism (i.e., the final patient-adapted perimeter of the implant selected does not impinge upon or otherwise violate the integrity of the locking mechanism). In alternative embodiments, the locking mechanism may extend along the entire superior surface of the tibial tray, whereby perimeter matching of the tray results in removal of some portion of the locking mechanism, yet the remainder of the locking mechanism is still capable of retaining the tibial insert on the tibial tray (i.e., the final patient-adapted perimeter of the implant impinges upon some of the lock structure, but sufficient lock structure remains to retain the insert in the tray). Such embodiments may have locking mechanisms pre-formed in a library of pre-formed tibial tray blanks As another alternative, one or more locking mechanism designs may be incorporated into the implant design program, with an appropriate locking mechanism design and size (including scaling of a design) chosen at the time of implant design, and ultimately formed into (or otherwise attached to) a tibial tray (chosen or designed based on the patient's anatomy) during the process of designing, manufacturing and/or modifying the implant for use with the specific patient. Such design files can include CAD files or subroutines of locking mechanisms of various sizes, shapes and/or locking features, with an appropriate locking mechanism chosen at an appropriate time. Optionally, the design program can ultimately analyze the chosen/designed lock and locking mechanism to confirm that the final lock will be capable of retaining the insert within the tray under loading and fatigue conditions, and provide an alert (and/or choose an alternative design) if FEA or other analyses identifies areas of weakness and/or concern in the currently-chosen design.

Various embodiments of tibial trays described herein can include one or more cavities. The cavity or cavities (also referred to herein as tibial tray receptacles) can be designed for the tibial tray to receive a one-piece tibial insert or two-piece tibial inserts (or other quantities, as desired). The tibial tray may have patient-adapted cavity dimensions, including all patient-adapted dimensions or a combination of patient-adapted and standard dimensions. The cavity design, optionally in conjunction with integrated locking mechanisms, may include the ability to snap fit, press fit, interference fit, and/or have a mechanical fixation for the tibial insert. Additionally or alternatively, at least some portions of the cavity may be dimensioned to receive a portion of an insert with a clearance fit (i.e., the relevant dimensions of the cavity are slightly larger than the corresponding dimensions of the insert, such that related resistance to insertion of the insert is reduced or eliminated). Some embodiments may also include features (e.g., central rib or alignment guides, as discussed further below) that provide guidance for accurate orientation and placement of an insert into a cavity. Also, some embodiments may include configurations to provide audible signals or other indicators that can notify the surgeon that the insert is firmly fixed to the tray. In some embodiments, the cavities may include straight walls with no locking mechanisms, and may be dimensioned for an interference fit with a corresponding portion of the poly.

Additionally or alternatively, various embodiments can include tibial cavities configured for permanent fixation of the tibial inserts or configured with a mechanism for release of the insert. Permanent fixation may be accomplished by attaching the insert to the tray using mechanical means or the insert may be overmolded with the tray to create an assembly of the tray and the insert together. In an alternative design, the tray cavities may be designed to include one or more quick-release mechanisms to release the insert for insert size/thickness interchangeability. In various embodiments, the tibial tray may be designed to have a release mechanism that requires an additional tool so as to prevent or limit inadvertent release of the implant (or where the insert may be semi-permanent and/or require subsequent removal).

As discussed above, various embodiments of tibial tray cavities disclosed herein are designed to accept a tibial insert. The tibial insert may be designed as one-piece, two-piece, patient-specific, or a combination thereof, and there may be one or more cavities formed into a given tibial tray. For example, a tibial insert may use a patient-adapted profile to substantially match the profile of the patient's resected tibial surface. More specifically, the insert can be designed to match or optimize one or more features based on patient-specific data, such as a patient-specific perimeter profile and/or one or more medial coronal, medial sagittal, lateral coronal, or lateral sagittal bone-facing insert shapes or curvatures. The insert may be perimeter-matched to some or all of the tibial tray. In alternative embodiments, the tray perimeter may be undersized or the perimeter modified a desired amount to allow some rotation of the tray by the physician without significant overhang off the resected tibial surface.

In some embodiments, the tibial insert may also be uniquely designed to accommodate one or more locking mechanisms designed in the tibial tray. For example, if the locking mechanism is adapted to the patient's specific anatomy in at least one or more dimensions, the tibial inserts may be designed relative to the patient's articular anatomy and the dimensions or location of the locking mechanism may be selected and/or designed to desirably avoid or limit compromise of the locking mechanism. In various alternative embodiments, a tibial insert may be designed to incorporate an integrally-formed tab or other feature that engages into the locking mechanism to reduce or eliminate motion or rotation in order to reduce the potential for subsequent failure of the knee implant. The tibial insert may also have other constructs to engage with the locking mechanism (e.g., detents, tubes, screw attachments).

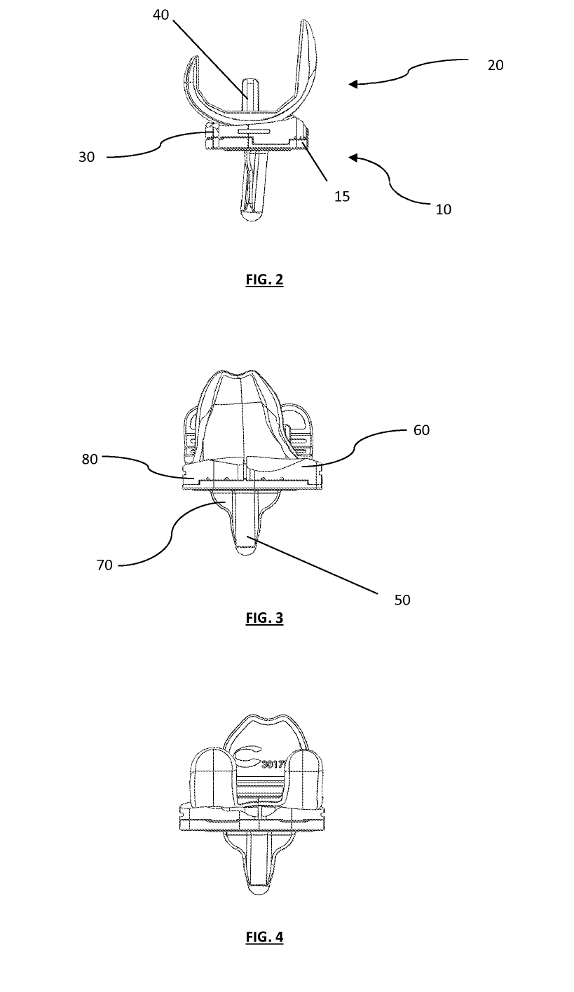

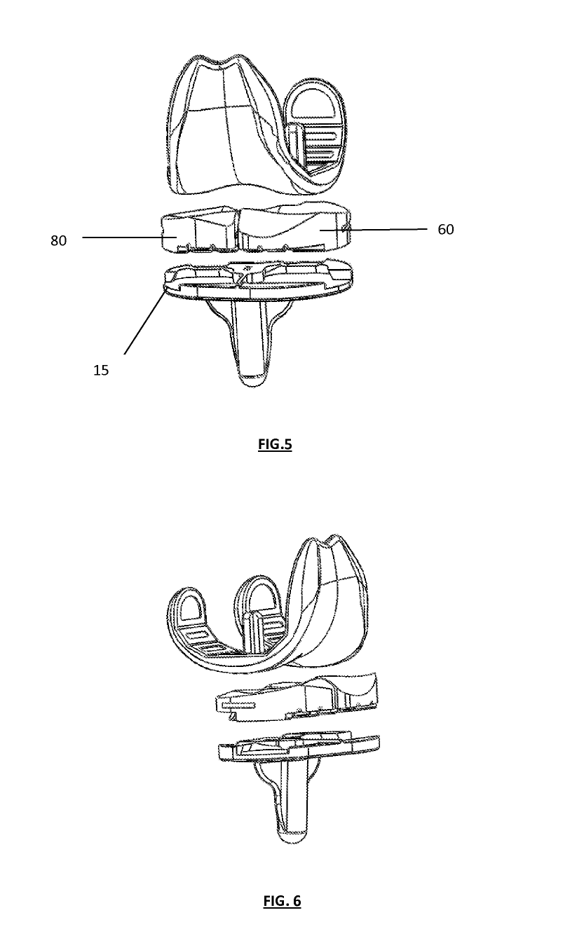

FIGS. 2 through 4 depict various side plan views of a total knee arthroplasty implant having a femoral implant component 20 and a tibial implant component 10. The femoral component 20 includes one or more anchors 40 for securing the component to a femur (not shown) of a patient. The tibial component 10 includes a tibial tray 15, a medial tibial insert 60, a lateral tibial insert 80, a tibial anchor 50 and one or more anchor stabilizers 70. FIGS. 5 and 6 depict exploded views of the knee arthroplasty implant.

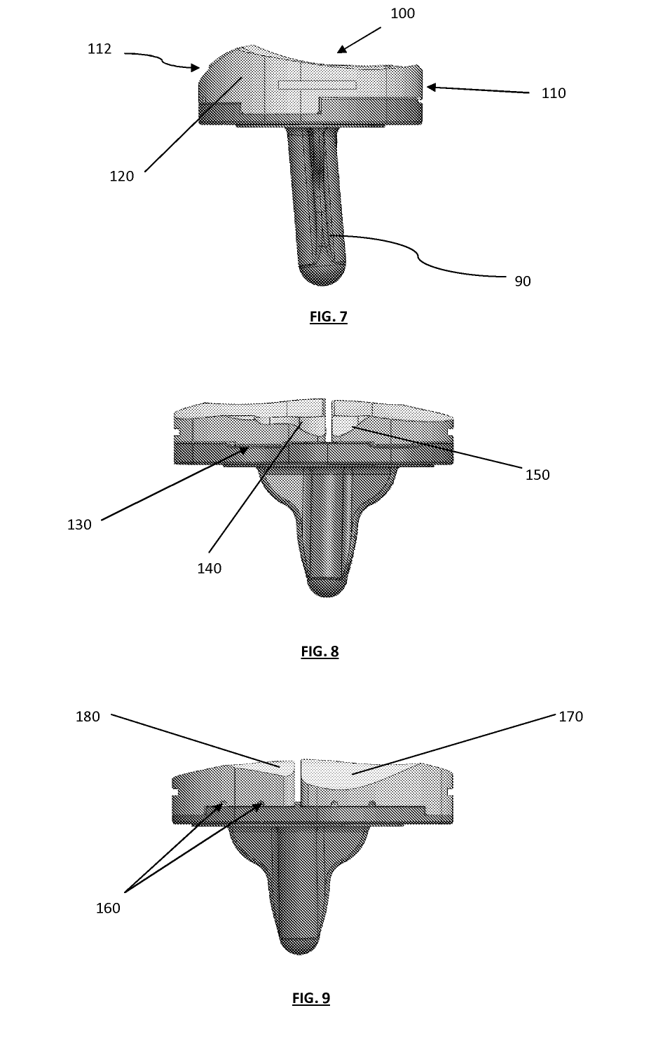

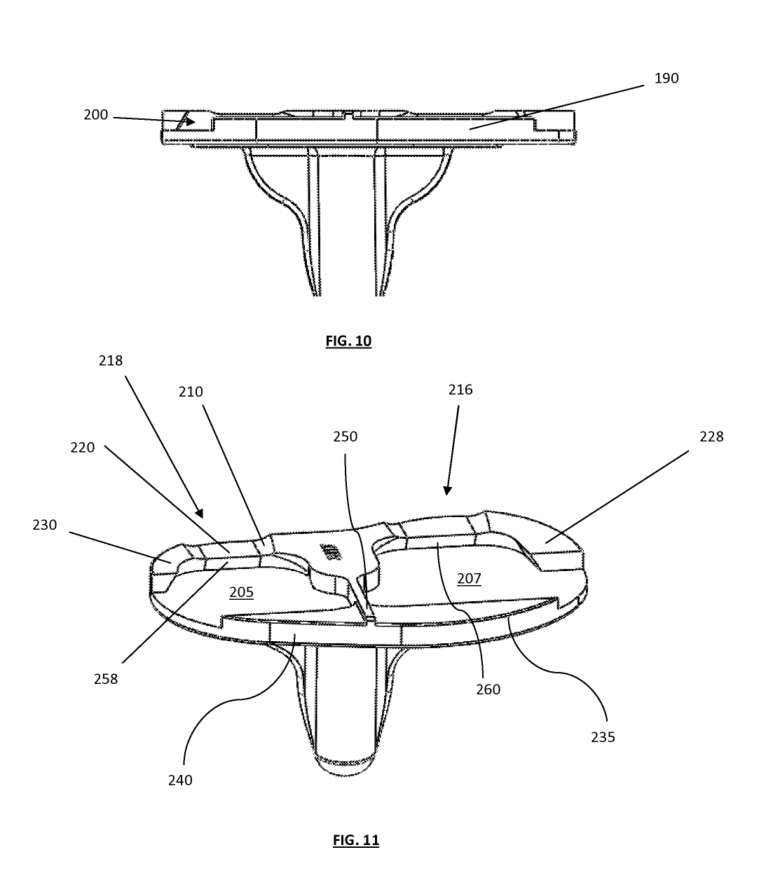

FIGS. 7, 8 and 9 show perspective views of an exemplary assembled tibial implant component. As best seen in FIG. 7, tibial inserts 60, 80 can include a posterior relief portion 112, an anterior portion 110, a side wall 120, a tibial tray stem 90, and an articulating surface 100. As shown in FIG. 8, medial tibial insert 60 can include a medial posterior soft tissue relief 140, and lateral tibial insert 80 can include a lateral posterior soft tissue relief 150, and a posterior guide 130. As shown in FIG. 9, medial insert 60 can include a medial anterior soft tissue relief 170, and lateral insert 80 can include a lateral anterior soft tissue relief 180. Such reliefs can be incorporated to accommodate various soft tissue structures, such as the various ligaments in the knee. In some embodiments, tibial inserts 60, 80 can include one or more anterior openings 160. Such anterior openings 160 may be configured to accommodate engagement of a release tool (not shown) to release a detent or other locking mechanism holding the insert to the tibial tray 15. Openings 160 and associated release channels may be formed into one or both of the medial 60 and lateral 80 inserts. Release channels may be configured to allow the surgeon to insert a standard release tool into one or more of the channels to release the locking mechanism and allow the tibial insert to be removed from the tray. Such channels can allow the surgeon to remove one tibial insert from the tray and replace the insert with another insert (e.g., an insert having a different thickness), as desired. In some embodiments, tibial inserts may be configured to be reattached after removal. In other embodiments, tibial inserts may be configured to prevent reattachment after removal. For example, inserts may be configured such that removal damages or otherwise alters the insert, eliminating the possibility of reattachment.

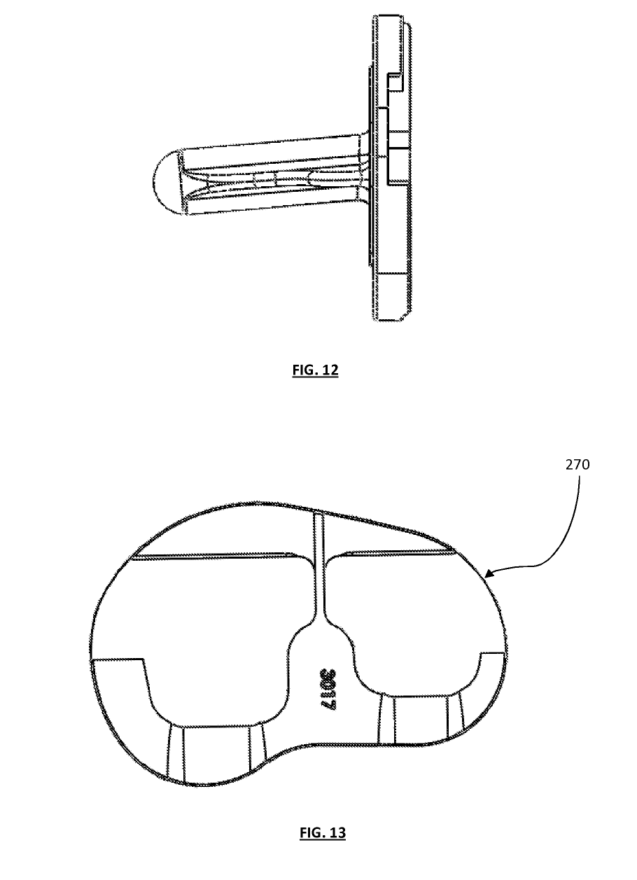

FIG. 10 depicts a front plan view of one embodiment of a tibial tray having an anterior wall 190 and a lateral wall opening 200. As best seen in FIG. 11, the tibial tray can include a medial cavity 207 and a lateral cavity 205. While this embodiment is designed to include a two-piece tibial insert, alternative embodiments may include design features that accommodate a one-piece tibial insert. The medial cavity 207 can include a medial anterior wall 235 and a medial posterior wall portion 260. The posterior wall 260 may have a flat, beveled or chamfered wall to allow engagement with the insert, as well as potentially make engagement of the tibial insert stronger and reduce micro motion of the insert. Similarly, the lateral cavity 205 may have a lateral anterior wall 240 and a lateral posterior wall 258. The tibial tray may be designed to include a central rib 250 between the cavities. This central rib 250 may include a higher height than one or both of the medial or lateral anterior walls 235 and 240, to facilitate guiding and insertion of the inserts. In various embodiments, the central rib can also potentially function to reduce insert motion perpendicular to the sagittal plane (i.e., towards the center of the tibial tray), and potentially reduce failure of the locking mechanism resulting from undesired insert rotation. Cavities 207 and 205 can further include peripheral walls 228 and 230, and alignment guides 216 and 218. In some embodiments, alignment guides 228, 230 can include a detent section 220 and one or more chamfered walls 210.

In some embodiments, the medial and lateral anterior walls 235 and 240 may be shorter than the posterior walls 260 and 258 and/or the central rib 250. Such a height difference may facilitate proper orientation and placement of inserts upon initial insertion. For example, as the insert is inserted into position, the insert structures can pass over the anterior wall and along the central rib (see FIG. 21). Upon further insertion, an alignment feature formed on an underside of the insert can be configured to slide into and along alignment guides 235, 240. The insert ultimately seating in and securing to the locking mechanism. Various structures described herein can facilitate posterior engagement of the tibial insert, which may make insertion of the tibial insert easier.

FIGS. 12 and 13 depict side and top views of the tibial tray of FIG. 10. As best shown in FIG. 13, the perimeter or shape 270 of the tibial tray can be designed using various methods to incorporate dimensions from many sources, including standard sized dimensions derived from a database of various patients tibia. Various dimensions may also (or alternatively) be derived from patient-specific data such as 3D images taken from a patient that is converted to image patient data. The image patient data can be used to measure the perimeter of the tibia and a patient-specific size may be manufactured.

FIGS. 14 through 16 depict various views of exemplary medial and lateral inserts. As can be seen in FIG. 14, the inserts can each include one or more peripheral grooves 280 formed in a peripheral edge of the insert. In various embodiments, such grooves may be used as contact and/or holding points for manufacturing purposes. For example, manufacturing may require that the tibial insert be held on the peripheral edge to allow machining or other processing of the surfaces of the insert, as well as to prevent damage to the surfaces of the insert. A variety of feature shapes and/or sizes may be included on the peripheral edge to accommodate a shaped manufacturing tool that will help fixate the device during further processing requirements by the manufacturer.

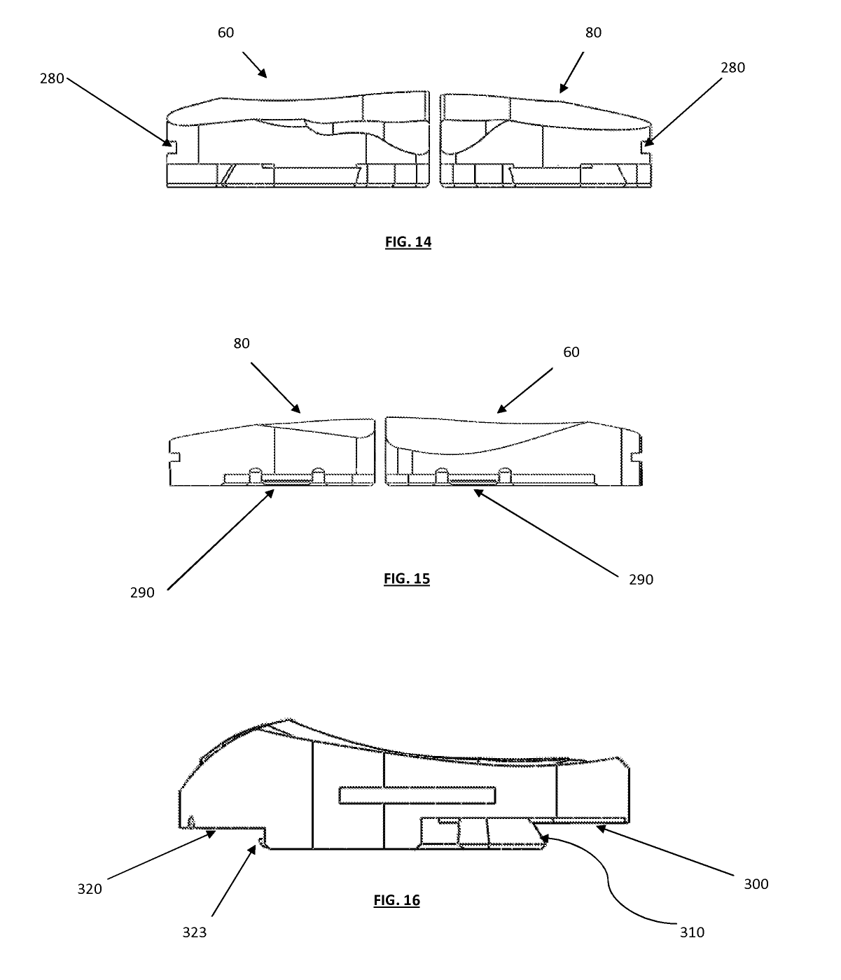

FIG. 15 depicts a front plan view of exemplary inserts. Each insert can include a detent tab 290. Detent tab 290 can include a portion that can latch or otherwise engage into an opening formed in the corresponding medial or lateral anterior wall of the tibial tray. FIG. 16 depicts a side view of an exemplary insert, showing an anterior cut portion 320, a posterior cut portion 300, and a posterior indent 310 formed in a lower surface of the insert, extending around a posterior portion of the insert. When the insert is secured to the tibial tray, the anterior indent 310 may accommodate a chamfered or dove-tailed surface of the posterior wall, securing the implant to the tray.

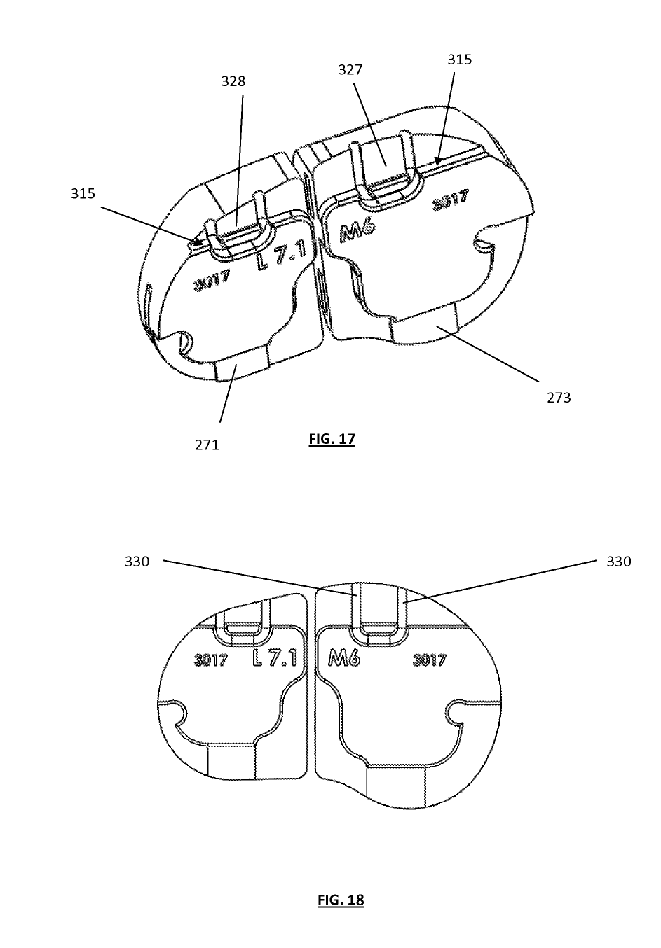

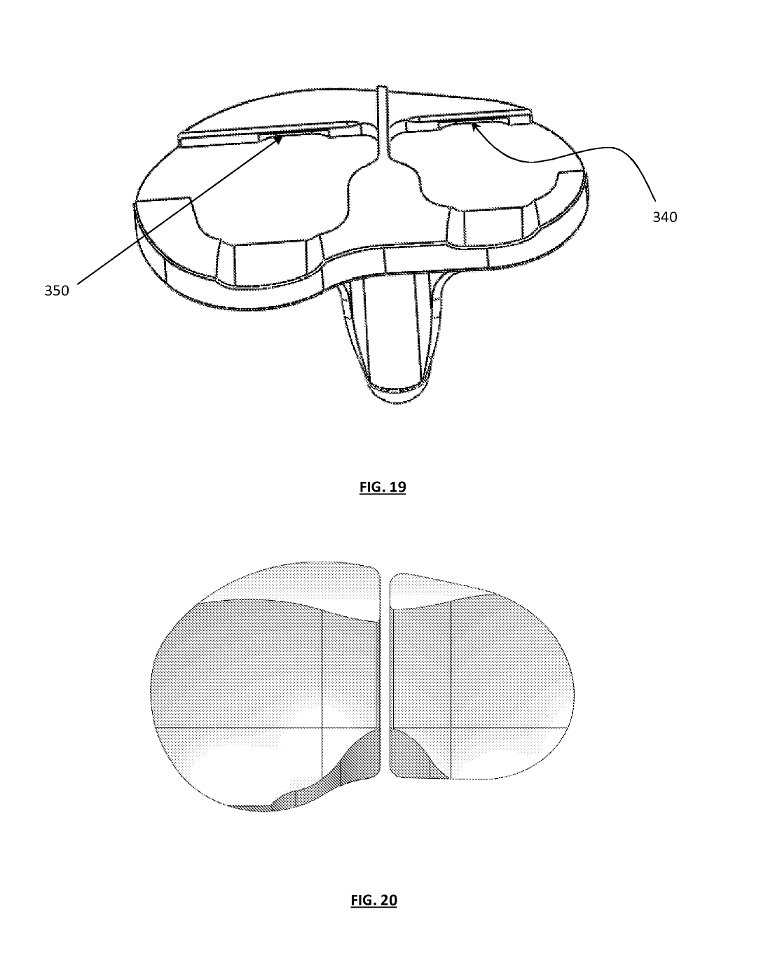

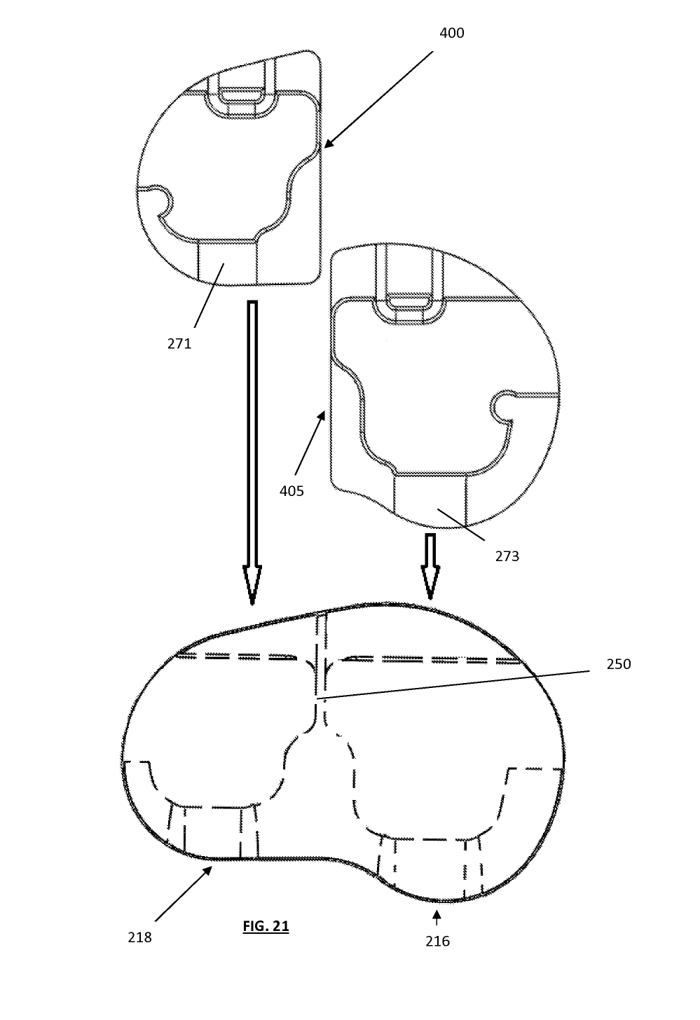

FIGS. 17 and 18 depict bottom perspective and plan views, respectively, of the exemplary inserts of FIGS. 14 through 16. Each of the inserts can include a planar anterior wall section 315 that corresponds to the medial and lateral anterior walls 235 and 240 of each respective cavity. Alignment tabs/detents 271 and 273 are formed in each of the inserts and can be configured to correspond to the detent sections 220 of the respective cavities (see FIG. 11). A detent tab 327 and 328 can be formed into the planar anterior wall section 315 of the anterior cut portions 320 of each insert. The detent tab can be surrounded by a cut portion 330, which can facilitate the flexion of the tab in a desired manner. A forward-facing protrusion 323 (see FIG. 16) can be formed into the anterior surface of each tab and can be configured to fit into a corresponding recess 340 and 350 formed into the anterior wall of each cavity 207 and 205 (see FIG. 19).

FIG. 19 depicts a perspective view of an exemplary tibial tray. The tibial tray can include a medial 350 and lateral 340 recess. Each recess may be designed to have a specific height, width and depth to improve the locking strength of the tibial insert into the tibial tray. Also, the recess may be designed to have a variety of shapes and/or dimensions that may be selected from various methods from many sources, including standard sized dimensions derived from a database of various patients' tibia. Various dimensions may also (or alternatively) be derived from patient-specific data such as 3D images taken from a patient that is converted to image patient data. The image patient data can be used to measure the tibia and a patient-specific sized recess may be manufactured, which may improve the strength of the locking mechanism.

In use of various embodiments described herein, each medial or lateral insert can be slid (separately or together, as desired) into the respective medial or lateral cavity of the tibial tray from an anterior to posterior direction (see FIG. 21). During insertion, inner walls 400 and 405 of each insert can optionally slide along and be guided by the central rib 250, which may facilitate aligning the insert such that the alignment tabs 271 and 273 are positioned such that they slide into corresponding alignment guide 218 and 216 formed in each of the cavities.

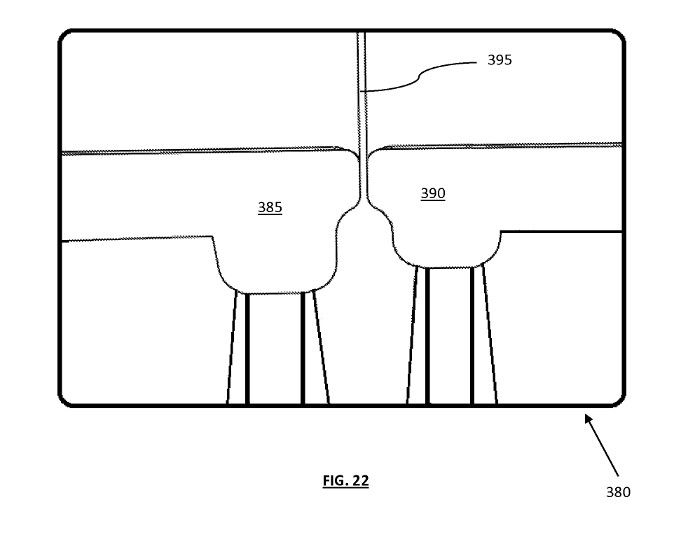

In various embodiments, one or more patient-adapted tibial trays can be prepared prior to the surgery, based on various patient-specific measurements. For example, a tibial tray blank 380, such as depicted in FIG. 22, can be selected from an inventory of pre-formed blanks, and the perimeter of the tray can be machined and/or otherwise processed to create a tibial tray suitable for use with a specific patient. The blank 380 can include a pre-formed and/or pre-machined pair of medial and lateral cavities 385 and 390, central rib 395, alignment guides and associated perimeter materials such that, when the blank is prepared, sufficient perimeter structures (e.g., anterior walls, posterior walls, etc.) remain behind to secure the insert, as describe above.

In use, a physician will prepare a tibial implant site by removing some portion of the tibial plateau, such as by resecting the tibial plateau approximately 2 mm deep, thereby creating a flat, planar cut across the entirety of the tibial plateau (although angled and asymmetric stepped cuts are contemplated by the disclosure). A tibial tray can then be chosen appropriate to the size of the resected surface. In various embodiments, the tibial tray may comprise a blank or other partially or pre-formed implant component, which is then processed (desirably prior to the surgery using patient anatomical data and one or more predetermined surgical plans) such that the periphery of the selected blank component is formed to approximate the periphery of the resected tibial surface.

Once a tray is selected and prepared, the tray can be implanted on the tibia, with the tibial anchor at least partially located within a medullary canal of the bone, and a lower surface of the tray in intimate contact with the resected surface of the tibia. If desired, bone cement or other biocompatible material may be positioned between the resected surface and the bottom surface of the tibial tray in a known manner, and the bottom surface of the tibial tray can incorporate cement pockets or other known features.

After tray insertion, one or more inserts of an appropriate size and/or shape can be secured to the tray as described herein. In some embodiments, the inserts can be inserted in an anterior to posterior direction, and each slides over the anterior wall of the respective cavity of the tray, and travel along the central rib until the respective alignment tab slides into and along the alignment guide of the cavity. Each insert may be further advanced posteriorly, and the posterior indent may accommodate the posterior wall. Once in a fully inserted position, the chamfered or dove-tailed surface of the posterior wall 258 or 260 can desirably mate with the surface of the posterior surface 310 of the insert, which can secure the posterior section of the implant to the tray 258 and 260.

When the posterior surfaces mate, the anterior surface of the spacer may desirably pass over and clear the inner anterior surface of the respective cavity, and the lower portion of the insert may “drop” into the cavity (or may be pushed downward by the surgeon or by an appropriate tool). This action will may flex the detent tab slightly in a posterior direction, and the tab will slide along the anterior wall of the cavity until it reaches the corresponding recess 340 or 350 formed into the anterior wall of the cavity, where the tab can flex slightly anterior and the forward-facing protrusion on the anterior surface of the tab will be captured by the recess, desirably securing the insert into the tray.

The capture arrangement of the locking mechanism, in combination with the central rib and various additional features of the design, may significantly increase the resistance of the insert to failure from various loading modalities, including significant posterior loading of the spacer. Once implanted into a patient, a tibial tray insert can experience significant axial loading (which can tend to push the spacer into the tray) and anterior/posterior loading (as the knee flexes and extends). Typically, the medial/lateral loading of a tibial spacer is more limited, as sideways loading and/or impacts to the knee and knee components are less common. Further, various features disclosed herein, including, for example, the central rib and alignment guides, may facilitate and/or improve the accuracy of insertion of the insert by a surgeon during the surgical procedure.

When posterior loads are experienced to a significant degree by the insert, some existing insert designs have allowed the spacer to separate from the tray and/or otherwise fail. In various present embodiments, however, the capture of the forward-facing protrusion may not only prevent the spacer from dislocating anteriorly (by “popping up” and off the anterior wall of the cavity), but may also prevent the anterior surface of the spacer from sliding or moving medial/laterally relative to the anterior wall of the cavity. This may not only prevent the spacer from sliding away from the central rib, but may also reduce the tendency for the spacer to flex and deform under extreme posterior loading, thereby reducing the opportunity for the spacer to dislocate.

What is claimed is:

1. A tibial implant, comprising:
a tibial tray configured for placement on a proximal surface of a tibia of a patient, the tibial tray including:
an inferior surface generally opposite a superior surface, a medial side generally opposite a lateral side, and an anterior side generally opposite a posterior side;
an anterior wall positioned on the superior surface and including at least a portion having a first height;
a posterior wall positioned on the superior surface posterior to the anterior wall, the posterior wall including at least a portion having a second height; and
a central rib positioned on the superior surface and extending generally posteriorly from the anterior wall, the central rib including at least a portion having a third height,
wherein the second height is larger than the first height.

2. The tibial implant of claim 1, wherein the anterior wall includes a posterior surface, the posterior surface facing generally posteriorly and including a medial portion and a lateral portion separated by the central rib, the medial portion of the posterior surface and the lateral portion of the posterior surface being generally coplanar.

3. The tibial implant of claim 2, wherein the medial portion and the lateral portion of the posterior surface of the anterior wall each include a locking mechanism, each locking mechanism configured to engage a portion of an insert.

4. The tibial implant of claim 1, wherein the posterior wall includes at least one alignment guide, wherein the at least one alignment guide comprises a depression in the superior surface of a portion of the posterior wall, such that the depression portion of the posterior wall has a height smaller than the second height.

5. The tibial implant of claim 1, wherein the tibial tray includes a medial cavity and a lateral cavity, wherein each cavity is positioned between the anterior wall and the posterior wall, the medial cavity is medial to the central rib and the lateral cavity is lateral to the central rib, and each cavity is configured to receive a portion of one or more inserts.

6. The tibial implant of claim 5, wherein the medial cavity is configured to receive a first portion of a first insert, and the lateral cavity is configured to receive a second portion of the first insert.

7. The tibial implant of claim 5, wherein the medial cavity is configured to receive a portion of a first insert, and the lateral cavity is configured to receive a portion of a second insert.

8. The tibial implant of claim 1, 2, 3, 4, 5, 6, or 7, including one or more inserts, wherein each of the one or more inserts includes a patient-adapted shape in at least one plane.

9. The tibial implant of claim 1, 2, 3, 4, 5, 6, or 7, wherein the third height is larger than the first.

10. The tibial implant of claim 1, 2, 3, 4, 5, 6, or 7, wherein the second height is larger than the third height, and the third height is larger than the first height.
